(19) United States
(12) Patent Application Publication
(10) Pub. No.: US 2007/0299315 A1
(43) Pub. Date: Dec. 27, 2007

(54) Novel retractor for hernia surgery

(76) Inventor: Peter L. Geller, New York, NY (US)

Correspondence Address: MORRISON & FOERSTER LLP, 1650 TYSONS BOULEVARD, SUITE 400, MCLEAN, VA, 22102, US

(21) Appl. No.: 11/471,720

(22) Filed: Jun. 21, 2006

(51) Int. Cl. A61B 1/32

(52) U.S. Cl. 600/217

(57) ABSTRACT

A hinged retractor for use in hernia repair, having: a handle portion to operate the retractor; a ratcheting device to releasably maintain the retractor in a plurality of open positions; a plurality of blades; and two arms formed such that they pivot around a hinge when the handle portion is operated; wherein each of the arms is formed such that at least one of the plurality of blades may be removably attached.

### BACKGROUND OF THE INVENTION

Inguinal hernia repair is one of the most commonly performed operations in the United States, with over 700,000 such repairs performed annually. It is estimated that twenty-five percent of males and two percent of females will develop inguinal hernias in their lifetimes. The vast majority of inguinal hernia repairs are performed through a skin incision which exposes the hernia defect in the inguinal canal of the abdominal wall.

To aid surgeons in visualizing the hernia defect through the skin incision, a hinged retractor is inserted into the incision. Once inserted, the retractor is opened to a sufficient width and then locked in position using an integrated ratcheting device. Even though hernias occur in a variety of sizes, types, and locations in the abdominal wall, surgeons performing hernia repairs presently rely on a small number of standard self-retaining retractors to assist in visualizing the hernia defects.

Adequate visualization of the abdominal wall defect is the main determinant of the width of a hernia incision. It is important to note, however, that hernia repairs frequently employ a mesh prosthesis that is placed in the incision and sutured over the defect. It is therefore important to adequately visualize the tissue around the hernia defect to assure accurate suture placement and a durable repair.

All of the available self-retaining hernia retractors are similar in design and have been in use for over fifty years without any substantial modification. The most commonly used of these retractors, which is employed for retraction in brain surgery as well as in hernia surgery, is known as a cerebellar or Adson-Beckman hinged-arm retractor, and is a hinged device having a handle end and a blade end separated by the hinge. The handle end has two loops which are used for the thumb and middle finger of the surgeon, and resemble the handle of a pair of scissors. The blade end has two arms, each of which has an integral blade formed at its end. The integral blades are formed so as to have either three or four prongs which project perpendicularly from the arms in a downward direction, with the downward direction being defined as the direction in which the prongs are to be inserted into an incision. The downward-most end of each of the prongs is generally bent away from the opposite blade such that when the retractor is opened, the bent portions of the prongs face the direction of motion.

The retractors in use today are formed of two solid bodies, wherein each body forms a loop, an arm, and a blade. Furthermore, one portion of the integral ratcheting device is formed on each of the solid bodies and is located on the loop side of the hinge.

One reason for the consistency of the devices is that surgeons generally prefer to use well tested devices that they are familiar with. The risk of accident, and the accompanying malpractice concerns, prevents the acceptance of surgical devices that differ substantially in appearance and use.

### BRIEF SUMMARY OF THE INVENTION

There has been a dramatic increase in obesity in the United States in the past 20 years. The Centers for Disease Control and Prevention (CDC) define obesity as a body mass index greater than or equal to 30, or 30 lbs overweight for a 5′4″ person. In 1991, 4 states had obesity prevalence rates of 15-19 percent, and no states had rates at or above 20 percent. In 2004, 7 states had obesity prevalence rates of 15-19 percent, 33 states had rates of 20-24 percent, and 9 states had rates more than 25 percent (no data available for Hawaii). In other words, in 2004, 42 out of 49 states had obesity prevalence rates at or above 20 percent, compared with 0 states in 1991.

The well-documented trend toward obesity in the United States has resulted in a growing challenge to surgeons performing inguinal hernia repairs. One of the consequences of the increasing prevalence of obesity is the inadequacy of the available self-retaining hernia retractors to afford adequate exposure of inguinal hernia defects. Using the currently available retractors, surgeons are often unable to visualize inguinal hernia defects through small skin incisions. In order to obtain adequate exposure of the deep abdominal wall using current instrumentation, increasingly large incisions need to be made, coupled with the use of multiple self-retaining retractors within the incision. Larger incisions cause a higher rate of wound complications, including greater postoperative pain, increased blood loss, an increased incidence of wound infections, wound dehiscence, and recurrent hernias.

In brief, currently available self-retaining retractors are equipped with blades that are insufficiently deep to afford adequate exposure of the inguinal canal in obese patients. As a result, larger incisions and additional surgical assistants or instruments are needed to facilitate exposure of the surgical field.

This problem is the result of the thickness of the fat layer in obese patients. Because an obese patient's subcutaneous fat layer is deep, the blades of currently available retractors are too short and/or too shallow to hold back the fat layer. Since the prongs of the retractors are not long or wide enough, surgeons have started to use two or more retractors inserted at different angles to attempt to hold back the fat and thereby achieve adequate wound exposure. Even when a surgeon is able to retract the fat a sufficient amount using multiple retractors, the operative field becomes cluttered with instrumentation and harder to operate in, thus increasing the difficulty of the repair as well as the risk of error. This problem extends beyond patients with morbid obesity. The average American patient is sufficiently large that the currently available retractors are not able to retract an adequate depth of fat. Even patients exhibiting lesser degrees of obesity present a problem for the currently available self-retaining retractors. What the CDC data show is that one in five patients in 42 out of 49 states will be obese by an even stricter, more extreme standard than the run-of-the-mill overweight patients. Stated another way, one in five patients will be morbidly obese while an even larger number will be sufficiently obese to render the current instrumentation useless.

For practical purposes, obesity, as used herein, is defined as a body fat thickness that exceeds the depth of the currently available self-retaining retractor blades. Accordingly, a patient with a fat layer of greater than 1.5 cm may be considered obese for the purposes of the invention.

Simply designing a retractor with longer or deeper blades will not address the problem. The depth of the subcutaneous fat layer (as well as the size of the hernia defect) varies widely among patients, both the overweight and the obese. Thus a device having fixed blades cannot be ideally adaptable. The ideal device would have exchangeable blades that can be chosen to allow the desired length and depth of retraction while the retractor's arms lie flush against the patient's skin.

Accordingly, while a retractor having a 10 cm blade would be very beneficial if the patient had 10 cm of fat, it would be unacceptable if the patient had a 2 cm or 20 cm of fat. While it would be technically feasible to stock every operating room with multiple retractors, with each reactor having a different size of blade, it would not be practical for several reasons. Stocking an operating room with multiple retractors would greatly increase the cost to hospitals which would have to acquire and maintain the retractors. Furthermore, increasing the number of devices in an operating room would increase the chance of devices and other surgical aids being misplaced or getting in the way.

Assessing the patient before surgery to determine which blade size should be used might minimize the number of tools brought into an operating room. However, surgeons have been unable to accurately determine the thickness of the fat layer of a patient until the incision is actually made, and after the incision is made any search for an appropriate sized instrument results in delay and increases the time the surgical wound is open and the associated complications and costs.

Accordingly, a retractor having removable blades has been developed. In one embodiment of the invention, the blades can be of various shapes and/or sizes, examples of which are detailed below. By using such a retractor having removable blades, a surgeon will be able to make an incision in the patient and then quickly determine, select, and attach the appropriate sized blades onto the retractor frame. The use of removable blades vastly increases the versatility and functionality of the retractor and will allow surgeons to visualize hernia defects without the difficulties associated with conventional retractors.

While self-retaining retractors with exchangeable blades are currently available for cardiac surgery, these retractors are not suitable for use in hernia repairs. The cardiac retractors are designed to mechanically separate the sternum and maintain exposure of the heart after the sternum has been split with a saw or knife. The force required to accomplish the task of sternal retraction is considerably greater than the force needed to separate the skin and subcutaneous fat of the abdominal wall over the inguinal canal because of the disproportionately greater recoil counterforce of the bony ribs which support the sternum compared with the merely elastic collagen of the skin. Consequently self-retaining sternal retractors are heavy, bulky instruments which utilize cog-wheel mechanisms strong enough to actually fracture ribs. In contrast, self-retaining abdominal wall retractors used for superficial surgery such as inguinal hernia repairs are low-profile light-weight instruments with simple ratchet joints required to maintain subcutaneous exposure with minimal force. Sternal retractors can not be operated using one hand and can often require a second person to hold the retractor in place while it is being operated.

There are, in addition, numerous self-retaining retractors available for use in deep intra-abdominal surgery, including the Bookwalter, Rochard, and Thompson retractors. However, these retractors are much too large and heavy for use in hernia repairs. Furthermore, they require fixation to the operating table to maintain stability, provide great force to overcome the recoil of the abdominal wall's full thickness musculature, and are equipped with large retractor blades that are unsuitable for the small operative field used in an inguinal hernia repair. These retractors are also unsuitable for use in a hernia repair in that they cannot be easily positioned around a patient to properly align with an incision.

In contrast to the sternal retractors and the deep abdominal surgery retractors, hernia retractors rest lightly on the patient's body and may be easily manipulated and positioned using a single hand. By allowing single hand operation, a surgeon is able to manipulate the retractor with one hand while manipulating the tissue surrounding an incision with the other hand, thereby improving the efficiency and accuracy of the placement of the retractor. Furthermore, because the retractor can be moved about the patient's body with ease, the location of the incision is not determined by the physical characteristics of the retractor.

In addition to the difficulties associated with the varying thicknesses of fat discussed above, each patient may present additional difficulties. For example, difficulties may arise that are associated with the consistency of fat, the shape of the patient's body, the length of the incision, the angle of the incision and/or the location of the defect in relation to the incision. Accordingly, various embodiments of the invention include using blades having different shapes as well as different sizes. The variations in shape may include, but are not limited to, at least: the number of prongs on the blades, the shape of the prongs, the number of barbs on each prong, the angle at which the blades are fixed onto the retractor arms, changing the angle to which the blades are attached to the arms such that the blades will be parallel to each other when the arms are opened a predetermined amount, the angle of the prongs in relation to the other prongs on the same blade, the angle of the prongs as they relate to the prongs on the opposite blade, pairs of blades where the blades are identical, pairs of blades where the blades are not symmetrical, blades having prongs of different lengths, blades having prongs of different shapes, and/or the angle of the prongs as they relate to the arms of the retractor.

Furthermore, various embodiments of the invention may include, but are not limited to, at least retractors in which: the arms are formed at different angles, such that they can lay flush with a patient's body while the edges of the blades are parallel with the hernia defect; and/or hinged arms that can be adjusted to change the angle of the blades in relation to at least a portion of the arms.

Additionally, various embodiments of the invention may include retractors in which the blades are attached to the arms using various types of attaching devices. These devices may include, but are not limited to, at least devices involving: snapping mechanisms; threaded members; resistively attachable couplings; and/or shaped couplers.

In one embodiment of the invention, the blades are sized such that they are almost the full length of the incision. In this embodiment, the blades do not extend to the corners of the incision; accordingly, the skin at the ends of the incision will not excessively restrict the opening of the retractor arms, but the blades remain long enough that they prevent tissue from collapsing in at either end of the incision.

In another embodiment of the invention, the retractor and a selection of blades are packaged in a single kit.

To simplify the surgeon's decision as to which removable blade to use, one embodiment of the invention includes a device to measure the depth of the fat and to indicate and identify the appropriate removable blade to be used. In a further embodiment of the invention, this device is a series of markings formed onto at least one of the retractor arms.

To further assist the surgeon, a scale may be used to estimate the approximate thickness of a patient's fat layer prior to making an incision. A ratio between measurable values, such as body mass index or fat percentage, and average thicknesses of the fat layers of patient's having similar measured values can be used to estimate the thickness of a patient's fat layer based on. The ratio is not limited to the above measured values, but can be based on any measured values that are shown to have a relation to the thickness of a patient's fat layer.

The length of the incision to be made is based mostly on the depth of the fat that needs to be cut through to gain exposure of the hernia defect. Better exposure allows for a smaller incision to be made. To aid a surgeon in determining the minimum length of the incision that must be made to adequately visualize the hernia defect, a further aspect of the invention involves marking a measuring device and/or the removable blades with an indicator that identifies the proper length of incision to be made. This indicator may indicate a length that is based on a predetermined ratio between fat layer thickness and minimum incision length.

According to one embodiment of the invention, optimal exposure of an inguinal hernia defect may be obtained through an incision having a length that is at least three times the depth of the subcutaneous fat layer. The depth of the subcutaneous fat layer may be estimated to a moderate degree of accuracy in most individuals by using a hand held caliper to span the thickness of the skin and fat that can be easily pinched between two fingers, measuring the gap between the caliper blades, and dividing the measured gap by two to obtain the estimated depth of the fat layer. The estimated depth of the fat layer may then be multiplied by three to obtain the recommended length for the incision.

To simplify the above process, in an embodiment of the invention wherein the depth of the fat layer is to be estimated as described above, a plurality of blade sets may be marked with a thickness corresponding to a measured caliper span, such that the surgeon can determine which blade set to use without having to perform any arithmetic functions, thus reducing the chance of mathematical errors. The blade sets may be further marked with a recommended incision length based on the length of the blades or the estimated depth of the fat layer. In a preferred embodiment the blades may be marked with a recommended incision length that is three times the thickness of the estimated depth of the fat layer.

According to further embodiments of the invention wherein the depth of the fat layer is measured, a measuring device and/or a plurality of blade sets can be marked with a recommended incision length based on the measured depth of the fat layer. In a preferred embodiment of the invention, the recommended incision length is three times the depth of the measured depth of the fat layer. In an additional embodiment of the invention, the length of an incision may be extended such that it is at least three times the thickness, or depth, of the fat layer. According to one aspect of the invention, this may be done after the thickness of the fat layer is measured.

### BRIEF DESCRIPTION OF THE DRAWINGS

FIG. 1 shows a hinged retractor according to the prior art.

FIG. 2 depicts a hinged retractor having mostly straight arms according to the prior art.

FIG. 3A shows a side view of the blades according to the prior art.

FIG. 3B shows a frontal view of the blades according to the prior art.

FIG. 3C shows an angled view of the blades according to the prior art.

FIGS. 4A-D depict a series of coupling mechanisms according to various exemplary embodiments of the invention.

FIG. 5 depicts a coupling mechanism according to an exemplary embodiment of the invention.

FIG. 6 depicts various blades according to various exemplary embodiments of the invention.

FIG. 7 depicts various arms according to various exemplary embodiments of the invention.

FIG. 8 depicts a bent retractor arm having a removable blade according to an exemplary embodiment of the invention.

FIG. 9 depicts a hinged retractor having a hinged arm 330 and a removable blade according to an exemplary embodiment of the invention.

FIG. 10 depicts a surgical kit containing a hinged retractor having removable blades and various blades according to an exemplary embodiment of the invention.

FIG. 11 depicts a surgeon making an incision into a patient.

FIG. 12 depicts a surgeon trying to visualize a hernia defect using retractors according to the prior art.

FIG. 13 depicts a surgeon using the arms of a prior art retractor in an effort to visualize a hernia defect.

FIG. 14 shows a surgeon using a retractor according to the invention to adequately visualize a hernia defect.

### DETAILED DESCRIPTION OF THE INVENTION

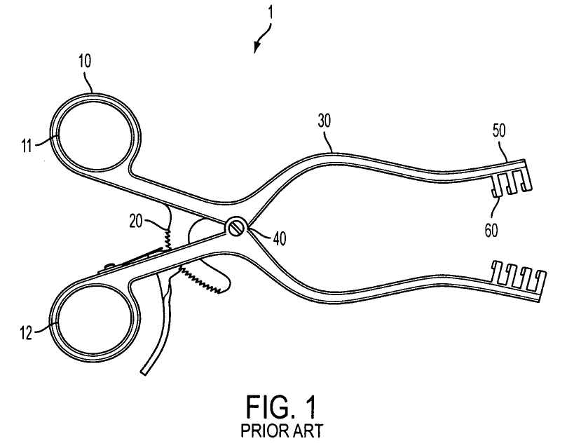

FIG. 1 shows a hinged retractor 1 according to the prior art. The hinged retractor 1 has a handle portion 10, itself having loops 11 and 12 to accommodate the finger and thumb of a user, respectively. The retractor 1 also has a ratchet portion 20 to maintain the position of the arms 30 when the retractor 1 is opened around a hinge 40. The arms 30 have blades 50 formed at the ends, and the blades 50 have prongs 60 extending therefrom in a downward direction.

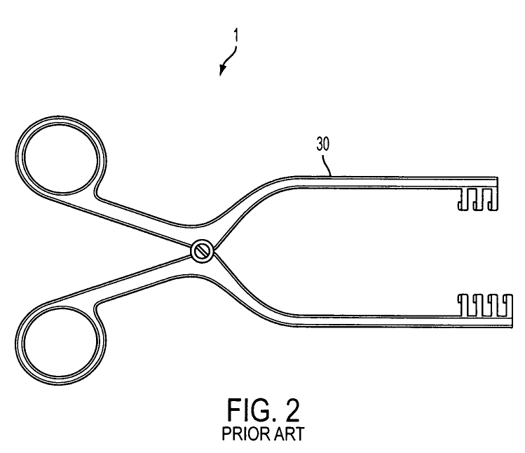

FIG. 2 depicts a hinged retractor 1 having arms 30 which are mostly straight as they approach the blades 50 according to the prior art.

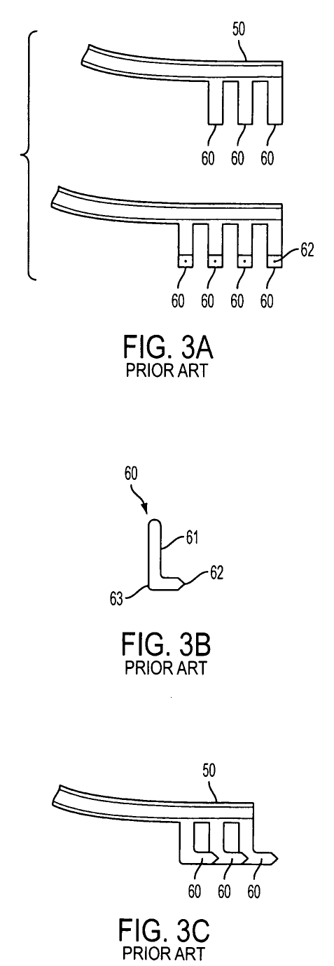

FIG. 3A shows a side view of the blades according to the prior art. The prongs 60 of the blades 50 are formed with barbs 62 (shown more clearly in FIG. 3B) that face outward from the retractor 1. The blades 50 are formed such that one blade 50 has three prongs 60 while the other blade 50 has four prongs 60. This formation allows the prongs 60 to be staggered such that they may overlap each other without interference when the retractor 1 is in a fully closed position.

FIG. 3B shows a frontal view of the blades according to the prior art. The prongs 60 are formed such that the barbs 62 extend at approximately a ninety degree angle 63 from the length portions 61 of the prongs 60.

FIG. 3C shows an angled view of the blades according to the prior art. The blades 50 are formed having prongs 60 that are all of an identical shape and orientation.

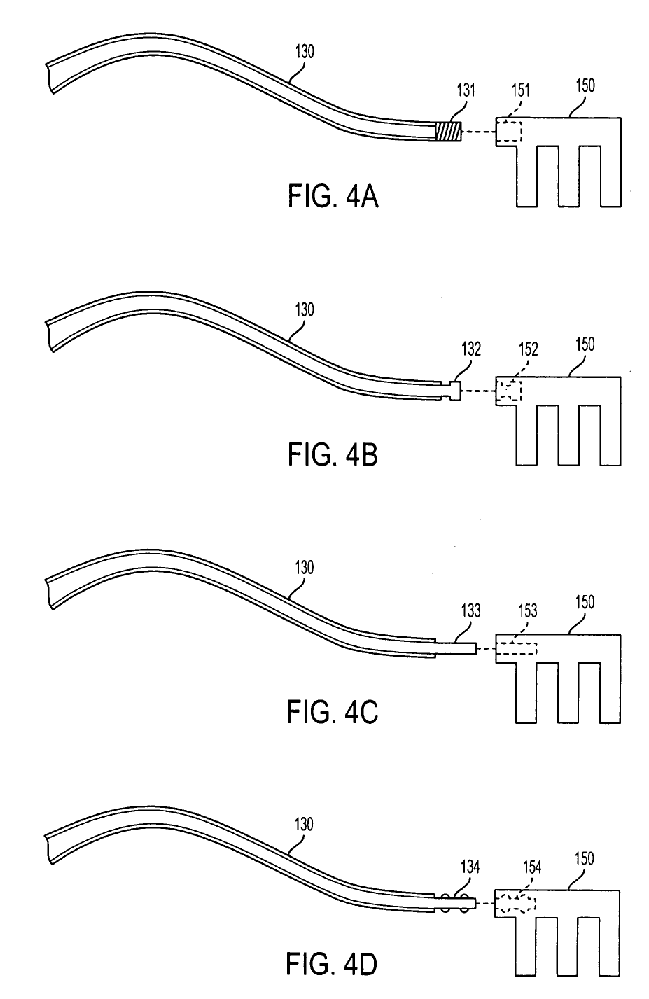

FIGS. 4A-D depict a series of coupling mechanisms according to various exemplary embodiments of the invention. The coupling methods shown are merely exemplary embodiments are do not in anyway limit the invention to the limited embodiments shown.

FIG. 4A depicts a retractor arm 130 having a threaded end 131 that can be mated with a threaded receptacle 151 of a retractor blade 150 according to an embodiment of the invention. A coupling device using threaded portions 131 and 151 has an advantage in that the blades 150 may be securely fastened to the arms 130.

FIG. 4B shows an embodiment in which an arm 130 has a knobbed portion 132 that can be inserted into a knobbed portion 152 of a blade 150. In at least one embodiment using a coupling mechanism with knobbed portions 132 and 152, the knobbed portions 132 and 152 have irregular shapes such that the blade 150 is restricted at least partially from rotating around the arm 130.

FIG. 4C depicts a retractor arm 130 having an end portion 133 which is inserted into a receptacle portion 153 of a blade 153. In at least one embodiment of the invention, the end portion 133 and the receptacle portion 153 have irregular shapes such that the blade 150 is restricted at least partially from rotating around the arm 130.

FIG. 4D depicts a retractor arm 130 that is formed having a shaped end 134 that can be fitted into a shaped receptacle 154 of a blade 150. In at least one embodiment of the invention, the shaped end portion 134 and the shaped receptacle portion 154 are formed such that the blade 150 is restricted at least partially from rotating around the arm 130. In various embodiments of the invention, the shaped portion 134 can be formed using a wide variety of shapes.

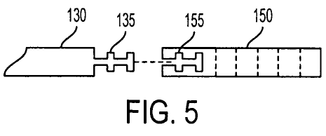

FIG. 5 depicts a coupling mechanism according to an exemplary embodiment of the invention in which a retractor arm 130 has a shaped end 135 that is formed to be fitted into a slotted end 155 of a blade 150. An advantage of this design is that the slotted end of the blade may be easily cleaned.

It is important to note that in alternate embodiments the coupling mechanisms can be formed in an opposite manner. For example the device of FIG. 5 could be formed with the slotted end 155 as part of the retractor arm 130 while the shaped end 135 could be formed on the blade 150.

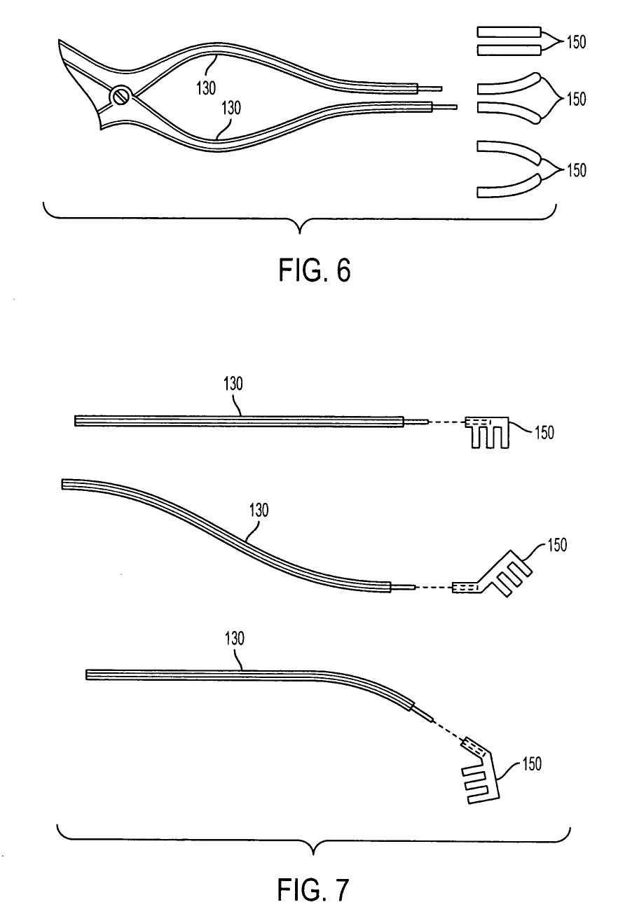

FIG. 6 depicts various blades according to various exemplary embodiments of the invention. As shown, the blades 150 can be formed in various shapes, each having an advantage according to the length and positioning of the incision. In one embodiment, the blades can be straight, while in others, the blades can be bent such that they bend towards or away from the opposite blade. The blades do not have to be uniform, and in some embodiments it may be preferable to use blades having different shapes and or sizes.

FIG. 7 depicts various arms 130 according to various exemplary embodiments of the invention. As shown, various embodiments of the invention use blades 150 having different mounting angles such that a surgeon can select a blade 150 that allows for maximum visualization of a hernia defect. In various embodiments of the invention, retractor arms 130 having different shapes may also be used. By using either the shaped arms 130 or the angled blade 150 alone or in combination, the surgeon may select a retractor that rests on a patient's body while positioning the blades 150 in an optimal position regardless of the patient's physical stature and/or the position of the incistion.

In alternate embodiments of the invention, the shaped blades 130 may also be removable with respect to either the retractor body, the blades 150 or both.

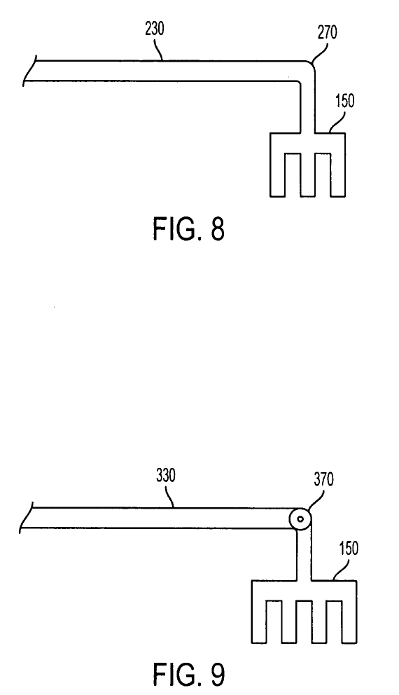

FIG. 8 depicts a bent retractor arm 230 having a removable blade 150 according to an exemplary embodiment of the invention. In various embodiments of the invention, the retractor arm 230 may be formed having a bend 270 such that the arm 230 can extend into the incision. A benefit of such an arm 230 is that a surgeon may be able to extend the arm further into an incision, for example with an extremely obese patient, while maintaining adequate visualization of the defect by minimizing obstructions such as extremely large blades.

FIG. 9 depicts a retractor having a hinged arm 330 and a removable blade according to an exemplary embodiment of the invention. As shown, the arm 330 has a hinge 370 which is formed to allow the surgeon to adjust the angle of the blade 150 with respect to the retractor, thereby allowing the surgeon more control over visualizing the defect.

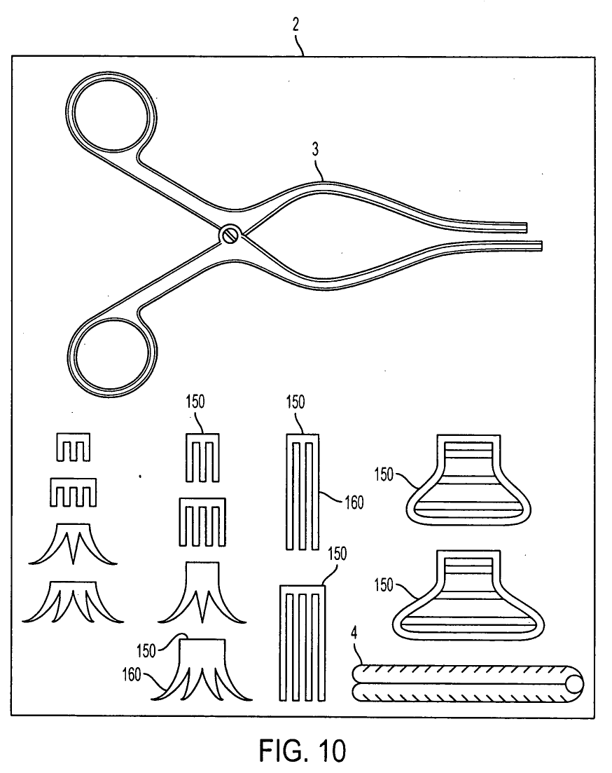

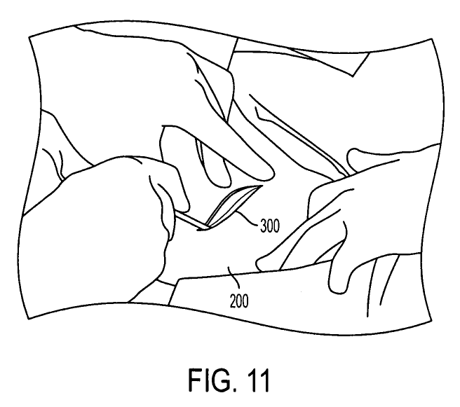

FIG. 10 depicts a surgical kit 2 containing a hinged retractor 3 and various removable blades 150 according to an exemplary embodiment of the invention. By packaging the retractor 3 in a kit 2 along with several different shapes and sizes of blades, a surgeon is provided with an endlessly customizable retractor. Although FIG. 10 only shows six pairs of blades 150, various embodiments of the invention are not in anyway limited to this number or assortment of blades 150.

FIG. 10 also shows a blade selection device 4 that may be used to aid the surgeon in selecting the appropriate blades 150. This device may be inserted into the incision to determine its depth, and may have marks that reference the appropriate blade to use according to the depth. In various embodiments of the invention, the blades are marked with their respective sizes. In various embodiments of the invention, the spaces in the kit are marked with the size of the blades according to where they are to be positioned.

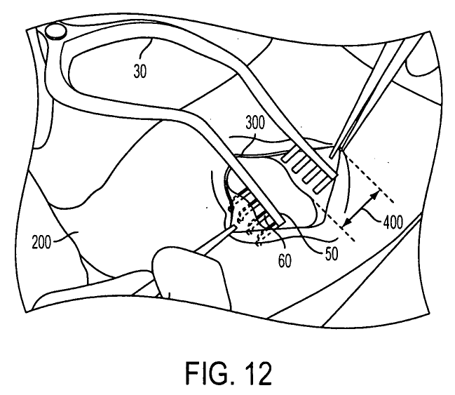

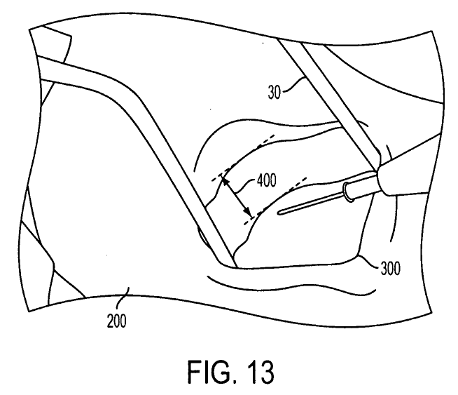

FIG. 11 shows a surgeon making an incision 300 into the abdomen of a patient 200. FIG. 12 shows the surgeon attempting to use a previously known retractor to visualize a hernia defect. As shown, when the blades 50 of the retractor are place into the incision 300, the length of the prongs 60 is less than the thickness of the fat layer 400. Accordingly, the prongs 60 only extend partially into the incision 300 such that the prongs 60 cut into the fat layer 400, causing damage to the surrounding tissue and not allowing for adequate visualization of the hernia defect.

To help compensate for the undersized blades 50, surgeons often resort to pushing the blades 50 farther into the incision 300 such that the arms 30 of the retractor are used to hold back some of the layer of fat 400 as shown in FIG. 13. Such misuse of the retractor can lead to extensive damage to the tissue surrounding the incision 300 due to the narrowness of the arms as well as the misalignment of the blades 50 and the prongs 60. Furthermore, the arms 30 of the retractor are insufficient to retain the fat layer 400, and appropriate visualization is not achieved.

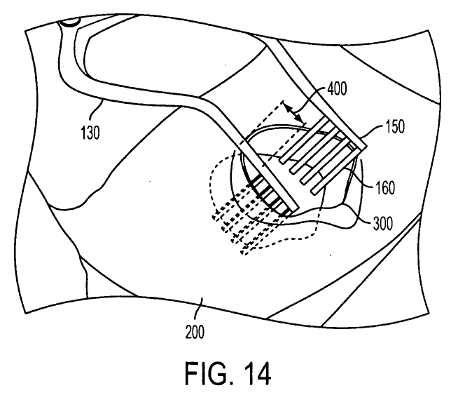

FIG. 14 depicts a surgeon using a retractor according to the invention to retract the fat layer 400. As shown, because the prongs 160 are sized according to the thickness of the fat layer 400, they may be inserted fully into the incision 300 without damaging the surrounding tissue. Furthermore, because the blades 150 are appropriately sized, the hernia defect may be adequately visualized, thereby reducing the patient's risk of complications.

Although various embodiments of the invention have been described herein, the invention is not limited by the embodiments described.

I claim:

1. A hinged retractor for use in hernia repair, comprising:
a handle portion to operate the retractor;
a ratcheting device to releasably maintain the retractor in a plurality of open positions;
a plurality of blades; and
two arms formed such that they pivot around a hinge when the handle portion is operated;
wherein each of the arms is formed such that at least one of the plurality of blades may be removably attached.

2. The retractor according to claim 1, wherein the arms are formed such that the blades may be removably attached to the ends of the arms.

3. The retractor according to claim 1, wherein the plurality of blades are formed in a plurality of sizes.

4. The retractor according to claim 3, wherein the blade sizes correspond to different thicknesses of fat layers.

5. The retractor according to claim 1, wherein the blades comprise prongs to retain fat.

6. The retractor according to claim 5, wherein the length of the prongs corresponds to differing thicknesses of fat layers.

7. The retractor according to claim 1, wherein the arms are hinged.

8. The retractor according to claim 1, wherein the blades may be removably attached to the arms at fixed angles.

9. The retractor according to claim 5, wherein a length of each of the plurality of prongs is at least 1.5 cm.

10. The retractor according to claim 5, wherein a length of each of the plurality of prongs is different.

11. The retractor according to claim 1, wherein the blades are marked with an indicator to identify a minimum incision length.

12. The retractor according to claim 11, wherein the indicator is based on a predetermined ratio between a fat layer thickness and incision length.

13. The retractor according to claim 11, wherein the minimum incision length is at least three times a depth of a fat layer.

14. A method of performing a hernia repair, comprising:
making an incision into a patient;
inserting at least a portion of a hinged retractor onto the incision; and
visualizing a hernia defect by opening the retractor,
wherein the retractor comprises,
a handle portion to operate the retractor;
a ratcheting device to releasably maintain the retractor in a plurality of open positions;
a plurality of blades; and
two arms formed such that they pivot around a hinge when the handle portion is operated;
wherein each of the arms is formed such that at least one of the plurality of blades may be removably attached.

15. The method according to claim 14, further comprising:
selecting blades from among the plurality of blades depending on a thickness of a patient's fat layer.

16. The method according to claim 14, wherein the thickness of the fat layer is determined after the incision is made.

17. The method according to claim 16, wherein the thickness of the fat layer is measured.

18. The method according to claim 16, wherein a length of the incision is extended to be at least three times the thickness of the fat layer.

19. The method according to claim 17, wherein a length of the incision is extended to be at least three times the measured thickness of the fat layer.

20. The method according to claim 14, wherein the plurality of blades are formed in a plurality of sizes.

21. The method according to claim 14, further comprising:
selecting blades from among the plurality of blades based on a predetermined ratio between a fat layer thickness and incision length.

22. The method according to claim 13, wherein the incision is at least three times the depth of a fat layer.

23. A surgical kit, comprising:
a hinged retractor comprising;
a handle portion to operate the retractor;
a ratcheting device to releasably maintain the retractor in a plurality of open positions;
a plurality of blade pairs, wherein each blade pair is of a different size; and
two arms formed such that they pivot around a hinge when the handle portion is operated;
wherein each of the arms is formed such that at least one of the plurality of blades may be removably attached,
wherein the plurality of blades are sized and arranged according to differing thicknesses of fat layers for which the blades are designed to retain.

24. The kit according to claim 23, further comprising a plurality of blades having different shapes.

25. The kit according to claim 23, further comprising a blade selection device to determine which of the plurality of blades is to be used on a patient.

26. The kit according to claim 25, wherein the blade selection device is to be inserted into an incision.

27. The kit according to claim 25, wherein the blade selection device comprises markings which indicate which of the plurality of blades is to be used depending on a depth of a fat layer.

28. The kit according to claim 25, wherein the blade selection device comprises markings which indicate a minimum incision length.

29. The kit according to claim 28, wherein the minimum incision length is at least three times the depth of a fat layer.
